(19) United States
(12) Patent Application Publication
(10) Pub. No.: US 2024/0389944 A1
(43) Pub. Date: Nov. 28, 2024

(54) SYSTEMS AND METHODS OF AUTOMATED BIOLOGICAL SIGNALS MONITORING USING PRICK NANOELECTRONICS

(71) Applicants: Agastya Brahmbhatt, Palo Alto, CA (US); Deepali Brahmbhatt, Palo Alto, CA (US)

(72) Inventors: Agastya Brahmbhatt, Palo Alto, CA (US); Deepali Brahmbhatt, Palo Alto, CA (US)

(21) Appl. No.: 18/796,289

(22) Filed: Aug. 6, 2024

(51) Int. Cl. A61B 5/00; A61B 5/145; A61B 5/1486

(52) U.S. Cl. CPC A61B 5/685; A61B 5/14532; A61B 5/14865

(57) ABSTRACT

The systems and methods comprising an automated prick nanoelectronics device with components: to select a seat of microneedles comprising of microneedles including: (i) a set of microneedles marked with one or more biomarkers of interest to the user; and (ii) number of set of microneedles included on the seat based on desired frequency of testing by a user; pop out the set of microneedles one at a time for testing based on the desired frequency of testing, to measure the biological signals based on the applied biomarkers on the microneedles in the set of microneedles and communicate the measured biological signals to a device selected by the user. Such a system could also work with a display or alert system and artificial intelligence-based analytics for the collected measurements of biological signals.

If an Application Data Sheet (ADS) has been filed on the filing date of this application, it is incorporated by reference herein. Any applications claimed on the ADS for priority under 35 U.S.C. §§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### FIELD OF THE INVENTION

The present invention is in the technical field of biological signals monitoring in a human body. The invention enables users to monitor, test and measure different biological signals of interest in a non-invasive manner using prick nanoelectronics based micro-needles in an automated manner at a customizable frequency in predictable time periods. The collected data from measuring biological signals is analyzed by an artificial intelligence module that can give further recommendations or feedback on testing to improve health.

### BACKGROUND

Medicine is no longer a one pill that fits all and is developing to be customized based on an individual's genetic makeup and bodily constitution. To support the understanding of how the body is performing, the importance of biological signals tests and measurements is also increasing. While there are some conditions, for example, blood glucose that can be continuously monitored, most of the biological signals including many protein signals associated with different health conditions cannot be monitored daily.

Even with the continuously monitored diabetes sensors, the sensors are hosted with a single needle deep inside the arm of the user for a period of 15 days to a month before it needs to be changed or moved locations within the body. Such permanent insertion for days has other potential side effects of infection and disease. The system is based on a user's diligence to be proactively clean and following instructions at insertion and removal of the diabetes sensor.

The testing of these biological signals using body sensors is expensive and dependent many times on insurance and underlying health conditions. While there are over-the-counter sensors and devices for athletic or general use purposes, those sensors do not measure the biological signals accurately.

Many of these sensors are also limited to testing one biological signal. A user may have multiple health conditions or desire to monitor multiple biological signals. There are no devices that provide this information to the user with a single sensor device.

Existing technologies and solutions do not work for several reasons. To get accurate biological signals, the needle needs to be inserted deep inside the tissue of a human body where the muscle is firm, for example, back of an upper arm or back of the upper leg. Such measurement is invasive and needs to be frequently changed in location, from one arm to the other arm to avoid causing infection at the point of insertion of the needle.

Non-invasive techniques, for example, sensing based on sweat of the skin often gives inaccurate results. Such measurements are too far out to provide a meaningful way of monitoring one's health. Not all health conditions can be monitored by sweat alone.

Frequent testing of blood is the only technique that can provide reliable test results that can be depended on for one's health decisions. None of the current techniques provide for an automated non-invasive manner of testing different biological signals using blood.

Moreover, the manual intervention of when and how to test is eliminated by automating the task of pricking or piercing for blood. The situation becomes even more challenging when a user is facing emotional or mental health issues or does not have a healthcare giver who is diligent in monitoring different biological signals.

For patients who are in the pre-disease, for example pre-diabetic stage, the onset of the disease or diabetes could be delayed or reversed by testing and measuring biological signals in a reliable manner without significant use of one's time and resources.

The problems with the existing sensors are most obvious for the blood sugar detection devices. The traditional blood sugar detection methods mainly comprise the following three methods: extracting venous blood for detection, blood sugar detection of a rapid blood sugar tester and blood sugar detection of a blood sugar test paper colorimetric method. However, these blood sampling measurements not only bring endless physical and mental double troubles to patients, but also are easy to infect and cause more serious consequences. Therefore, there is a necessary trend to develop a minimally or even non-invasive blood glucose concentration measuring system to alleviate the pain of diabetic patients.

Some of the continuous glucose monitors use a needle that collects information from interstitial fluid but requires the needle to stay inserted in the human body for a period of 15 days to a month. This increases the likelihood of infection at the spot of insertion.

The smart watches that claim to monitor glucose depend on sweat. Such data is not reliable because the sweat of the human body is not the same as the blood of the human body that has the most accurate data of what is happening in the human body.

While the market is flooded with wearable devices, none of them provide reliable and accurate biological signal monitoring that is non-invasive, regular and reliable. Such devices can be comfortably worn by a user and serve to extend perception, monitor status, and improve work efficiency. Among the existing clinical medical laboratory detection technologies, the biosensing technology is gradually a new research hotspot in the medical field due to the advantages of simple and convenient operation, high sensitivity, rapid analysis, small size, easy carrying, low cost and the like.

The biosensor technology mainly applies a microelectrode sensor, and the nano material plays an important role in the preparation of the biosensor due to the characteristics of good adsorption capacity, large specific surface area, many surface-active sites and the high catalytic efficiency. Yet, the biosensor technology with nano microneedle has not solved the problem of regular non-invasive monitoring that allows the skin to heal with minimal risks of infection.

Many types of wearable blood glucose meters from Dexcom and Abbott are available in the market. However, in all of them the acquisition of the biological signal is done using an insertion deep into the interstitial fluid level with a sensor that expires, and the location of insertion needs to be changed at frequent intervals to prevent damaging the tissue or causing an infection at the spot of insert.

### SUMMARY OF THE INVENTION

The present invention is systems and methods of a prick nanoelectronics device with sets of microneedles having different biomarkers (or antibody solutions) for biological signal testing that are pushed on for testing at a predetermined interval. The mechanics of intermittent regular non-invasive testing provides dependable and reliable biological signals measurements. The measured biological signal is collected for analytics and further used to configure the frequency and time periods of testing. The seat of microneedles is divided into days of the month and the number of rows included are based on the frequency of testing in a day. The automated system calculates the position to be pushed through the elastic buffer or spring mechanism at a given time and day of the month for testing. After testing is completed, the set of microneedles are pulled back allowing the pierced skin to be healed. For a given seat, the same position is not pierced for at least a month. This allows sufficient time for the skin to recover from piercing. This significantly reduces the risk of infection at the spot of piercing.

The systems and methods comprising an automated prick nanoelectronics device with components: selecting a seat of microneedles comprising of microneedles including: one or more set of microneedles marked with one or more biomarkers of interest to the user; and number of set of microneedles included on the seat based on desired frequency of testing by a user; popping out the set of microneedles one at a time for testing based on the desired frequency of testing; measuring the biological signals based on the applied biomarkers on the microneedles in the set of microneedles; communicating the measured biological signals to a device selected by the user.

The systems and methods comprising an automated prick nanoelectronics device with components, further comprising: indication of status of different sets of microneedles that have been used.

The systems and methods comprising an automated prick nanoelectronics device with components, wherein the user can revise the set of microneedles with different biomarkers using different needle length and size based on user's health or interest.

The systems and methods comprising an automated prick nanoelectronics device with components, further comprising: displaying the measured biological signals in a format based on user's choice including tabular, graphical, 2D, or 3D formats.

The systems and methods comprising an automated prick nanoelectronics device with components, further comprising: alerting the user when any of the biological signals fall outside of a desired range for the user.

The systems and methods comprising an automated prick nanoelectronics device with components, wherein: the popping out of the set of microneedles uses programmable elastic buffer mechanism, nanoelectronics on-off switch or molecular technology switches.

The systems and methods comprising an automated prick nanoelectronics device with components, wherein: the popping out frequency is synchronized with the time stamp on the user's device including a smartphone, smartwatch, smart tablet, laptop, computer or a cloud service.

The systems and methods comprising an automated prick nanoelectronics device with components, wherein the user can replace the seat of microneedles and the number of microneedles after use.

The systems and methods comprising an automated prick nanoelectronics device with components, further comprising: popping back in of the used set of microneedles.

The systems and methods comprising an automated prick nanoelectronics device with components, further comprising: artificial intelligence-based analytics on collected biologic signals that recommends corrective steps to promote good health.

### BRIEF DESCRIPTION OF THE DRAWINGS

The embodiments of this invention are illustrated by way of example and not limitation in the figures of the accompanying drawings, in which like references indicate similar elements and in which:

FIG. 1 shows a diagram illustrating an example of systems and methods of the invention, the prick nanoelectronics device with a seat of microneedles for measuring different biological signals communicating the measured data to types and categories of computing devices to perform analytics and give recommendations or feedback for health.

FIG. 2 shows the seat of microneedles divided uniformly to host different sets of microneedles, according to one embodiment.

FIG. 3 is a view showing one set of microneedles pressed for testing, according to one embodiment.

FIG. 4 is a view of customizing the prick nanoelectronics device and using the invention, according to one embodiment.

FIG. 5 is a view of customizing a set of microneedles that can be repeated or varied on the seat of microneedles for the prick nanoelectronics device, according to one embodiment.

FIG. 6 is a view showing design using components for the prick nanoelectronics device, according to one embodiment.

FIG. 7 is a flow diagram of method of using the prick nanoelectronics device, according to one embodiment.

FIG. 8 is a schematic diagram of exemplary computing devices that can be used to implement the methods and systems disclosed herein, according to one embodiment.

### DETAILED DESCRIPTION OF THE INVENTION

The systems and methods of prick nanoelectronics device with components includes techniques to customize a set of microneedles based on a user's interest or health conditions that can be repeated at a frequency required. The design or layout of the set of microneedles that can be used with the device is customizable and interchangeable. As a user's health changes or interest changes the set of microneedles can be changed and configured to measure different biological signals. At the end of a time period, for example, a month, the microneedles can be replaced for a new set of microneedles. For example, if a user is not testing many biological signals, he or she could have a seat of microneedles that can be used for a period of 3-months before replacement. On the other hand, if a user must monitor many biological signals, the user may need to replace the seat of microneedles more frequently in 2-weeks or even 1-week. The invention allows for flexibility in the period of testing and the number of biological signals to be tested according to a user's need. The user can replace the seat of microneedles and the number of microneedles after use.

The solution includes electronics to communicate the measured biological signals' data to a user's computing device including laptops, personal computers or smart watches. A user with a subscription to a service on the cloud could further transmit this data for analytics. In one embodiment, the analytics could include artificial intelligence analysis of the biological signals to predict future trends, patterns and identify any underlying health conditions that were previously not diagnosed.

The invention takes the hassle of monitoring out of the picture. For example, a user no longer needs to put in manual timers or alerts to measure the biological signals on time. The prick nanoelectronics device will do that with robotic accuracy and does not require manual intervention that could be subject to errors and mistakes.

The systems and methods of using prick nanoelectronics device for monitoring biological signals in a user, includes a repeated and predictable time interval pricking of the skin to collect droplets of blood and measure biological signals at that time in the user's body indicative of the body's signal concentration. It includes a device that receives and records the measured data from the prick nanoelectronics device.

In one embodiment, the device includes measuring and monitoring only a single biological signal, for example, blood glucose. The device is configured to display only single biological signal measurement values, and not any other measurement values associated with data received from the nanoelectronics device. In one embodiment, the data received from the prick nanoelectronics device is used to provide alarms to the user when the biological signal range or the rate of change of biological signal, as measured by the prick nanoelectronics device, is above or below a predetermined range.

Data received from the prick nanoelectronics device may also be used to prompt the diabetic or caregiver to take certain actions, such as take medication or perform another measurement.

In another embodiment, the device provides for toggling between two modes, with one mode that allows for display of biological signals measured and a second mode that prevents the display of biological signals.

In one embodiment, the display used is that of the user's computing device that already exists including smartphone, smartwatch, personal computer, laptop etc. In one embodiment, the alerts or alarms generated when a biological signal is out of predetermined range includes sending an email to caregivers or guardians. The alerts or alarms can also be configured to be excused for single or fewer occurrences and transmitted when they are out of predetermined range for multiple times for a continuous period beyond a threshold. For example, a single high blood glucose signal may not rise to the threshold of alarm, but repeated high blood glucose signals out of a predetermined range would be risen to an alert. In one embodiment, there would be levels of alarms from low to high depending on the severity of the biological signals measured data.

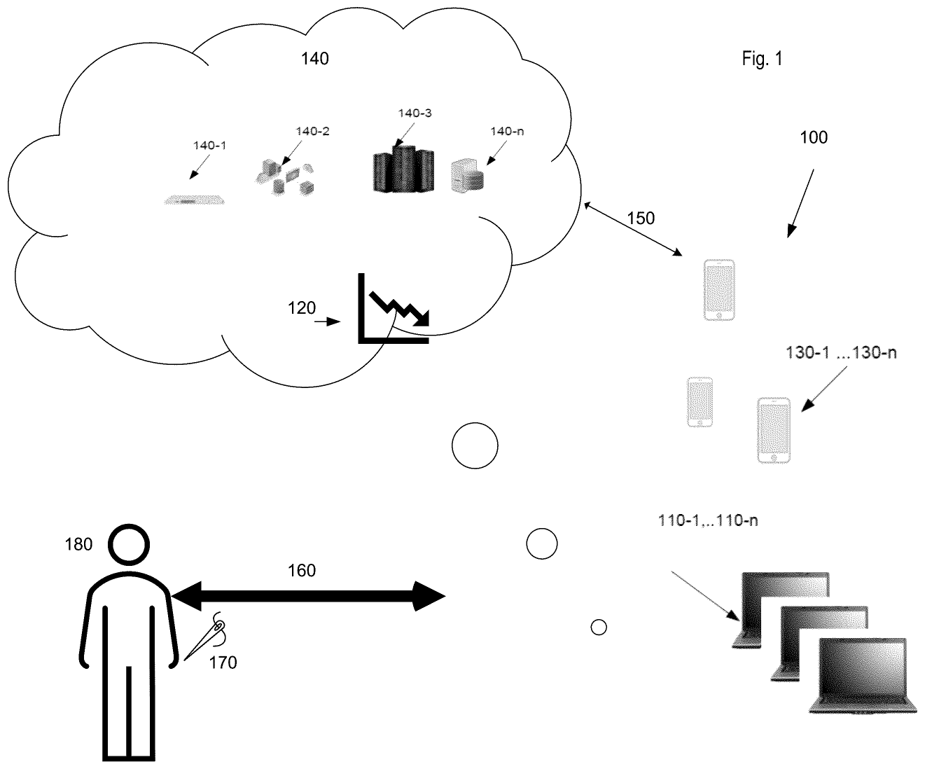

FIG. 1 shows a diagram illustrating an example of systems and methods systems of the invention, the prick nanoelectronics device, with different types and categories of computing devices including analytics. In one embodiment, as depicted in FIG. 1 different types of computing devices 100 connect to the prick nanoelectronics device 170 on the user 180. In the example of FIG. 1, different possible devices have been shown 110-1 through an nth client system 110-n, smartphones from 130-1 to 130-n, servers from 140-1, . . . 140-n. The prick nanoelectronics device 170 transmits data of measured biological signals using wireless transmission with the channel 160 to any of the computing devices, 110-1 . . . 110-n, 130-1 . . . 130-n, or 140-1 . . . 140-n. The collected data could be further sent for analysis in the cloud as shown by channel 150. Such analytics could be graphically displayed in reports 120. Reports on the measured biological signals could be in a format based on user's choice including tabular, graphical, 2D, or 3D formats. In one embodiment, the analytics could give feedback and recommendations to the user based on the biological signal measurements in his or her body.

In an implementation, prick nanoelectronics device, system and methods 100 may be accessible on the internet through secure protocols and strong authentication, authorization and encryption systems and processes. In an example implementation, 100 or any part of it can be inside a secure healthcare provide network or it can be in one or more cloud environments (public or private).

The prick nanoelectronics device is used for individual users with measured biological signal data that can be provided, analyzed and used by other cloud-based services, applications and software components.

A person of ordinary skill in the art would appreciate that by integrating data collected by the prick nanoelectronics device with existing or new healthcare or activities management software and systems, the deployment can assist users in actively manage their health in a hassle-free manner leveraging the advantages of real-time data and monitoring. While the invention does not provide for continuous monitoring, the interval of monitoring can be adjusted based on a user's health conditions and needs.

There are state of the art technology and different options to connect body sensors with computing devices to provide alarms, alerts or notifications. A person of ordinary skill in the art could use internet of things application programming interface on the invented prick nanoelectronics device here to connect with different cloud servers, software and applications. There are also solutions that are seamless and sensor to cloud solutions for a range of applications that can be incorporated in the invention to achieve transmission of prick nanoelectronics device data. The same mechanism can be used for duplex communication including giving feedback to the prick nanoelectronics device on data collection parameters.

The messaging and notification between different components can be implemented using application programming interface (API) calls, Javascript, extensible markup language (“XML”) or Javascript Object Notation (“JSON”) config file interfaces between different interfaces, Hypertext Preprocessor (earlier called, Personal Home Page) (“PHP”), Python, Node.js, Java/C++ object-oriented programming or web-based tools.

Different components may also implement authentication, authorization and encryption to keep the data and the requests secure. Authentication of a device, user and service may be accomplished using public/private key, passwords, token, transaction, biometrics, multi-factor authentication or other methods known in the industry. Encryption may use data encryption standard (DES), TripleDES, RSA, Advanced Encryption Standard (AES) or other cryptographic methods known in the industry.

The invention provides a customizable template on what biological signals need to be tested and at what frequency that are tailored for a user's needs. The invention gives the control back to the user to understand what is happening in his or her own body.

The invention relates to a wearable prick nanoelectronics device, which comprises a main body unit wearable on a human body that is a seat of microneedles with different sets of microneedles multiplexed for different biomarkers (or antibody solutions) to measure different biological signals at intermittent or regular frequency based on a user's needs. The seat of microneedles is broken into smaller sets of units, for example smaller rectangles. Each rectangle can be pushed and pulled at different times independent of the other rectangles at a time. When a rectangular unit with a set of microneedles is pushed, the microneedles pierce the skin and collect blood samples that are used to measure the biological signals. The push and pull mechanisms for each rectangular units could use programmable elastic buffer mechanism, nanoelectronics on-off switch or molecular technology switches.

A person of ordinary skill in the part would understand that the measuring of biological signals can be accomplished using well-established techniques. For example, a detection unit arranged on the device with a digital/analog circuit conversion module would be able to measure the biological signals accurately.

The device itself may or may not include a display. The device could optionally use the display of an existing user smartwatch, smartphone, laptop or personal computer.

A person of ordinary skill in the art would understand that the pushing of the rectangular unit with set of microneedles could be timed based on the required limits of time for a given biological signal and corresponding blood collection.

For example, for measuring glucose, a nano-micro-needle would include a micro-needle body and a silk fibroin layer wrapped outside the micro-needle body that also includes glucose oxidase which is exposed outside the silk fibroin layer change of which can be measured and would correspond to the glucose in the blood sugar droplet from the pricked microneedle.

The term biomarker or antibody solution as used herein, is a broad term, and is to be given its ordinary and customary meaning to a person of ordinary skill in the art (and is not to be limited to a special or customized meaning), and refers to without limitation to a substance or chemical constituent that reacts with a substance or chemical constituent in a biological fluid (for example, blood, interstitial fluid, cerebral spinal fluid, lymph fluid, urine, sweat, saliva, etc.) that can be measured. The biological signals can include naturally occurring substances, artificial substances, metabolites, or reaction products. In some embodiments, the biological signal for measurement by the sensing regions, devices, and methods is glucose. However, other biological signals are contemplated as well, including, but not limited to: acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphateuridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus,

Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute substances or chemicals that trigger biological signals in certain embodiments. The biological signal is proportional to the quantity of the substance or chemical that could be naturally present in the biological fluid or endogenous, for example, a metabolic product, a hormone, an antigen, an antibody, and the like. Alternatively, the substance or chemical can be introduced into the body or exogenous, for example, a contrast agent for imaging, a radioisotope, a chemical agent, a fluorocarbon-based synthetic blood, or a drug or pharmaceutical composition, including but not limited to: insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated substances or chemicals that produce biological signals on detection. Substances or chemicals such as neurochemicals and other chemicals generated within the body can also be analyzed using biological signals, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), and 5-hydroxyindoleacetic acid (FHIAA).

A person of ordinary skill in the art would understand that there are well-established

techniques to push and pull small units of areas in a larger seat of body, for example, here the seat of microneedles that has small units of sets of microneedles. For example, an elastic buffer element could be used to push or pull at a given location with the seat of microneedles. A small set of nanoelectromechical contact switches could be used to operate with different areas of the switch. The location of the push would be calculated based on the day of the month and time of the day. A module that works with the time on the internet or user's smartphone or smartwatch would sync the time on the prick nanoelectronics device. One embodiment could also use molecular technology to use small nano parts to press against the skin of the body for measurement and then pulled back.

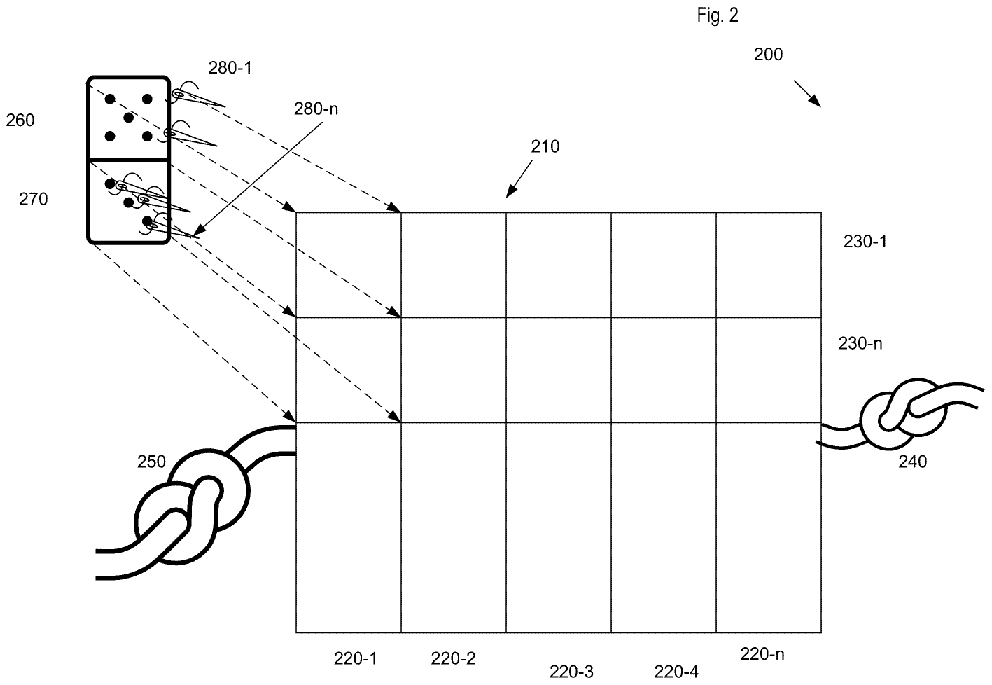

FIG. 2 shows the seat of microneedles 200 divided uniformly to host different sets of microneedles, according to one embodiment. FIG. 2 shows an example of a wearable device with straps 250 and 260. The rectangular unit 210 is the seat of the microneedles with the smaller set of rectangular units that have the row position from 230-1, . . . to 230-n depending on the number of times in day the microneedles need to be pushed for testing. The column positions from 220-1, 220-2, . . . to 220-n are based on the number of days in a month the seat of microneedles is to be used. For example, on day 10 of the month, the rectangular unit that would be used for testing would be at 220-10 column. The first instance on day 10 would use 220-10 column, 230-1 row position. The second instance of testing on the same day, day 10 would use 220-10 column and 230-2 row position.

In one embodiment, the biological signals tested in the first instance on day 10 could be same or different from those tested on second instance on day 10. For example, 260 is zoomed in version of the set of microneedles that show that it has a set of five microneedles. Whereas the second set of microneedles at 270 shows only three microneedles. Each unit can be configured based on a user's needs.

The set of needles 280-1, . . . to 280-n are on a small thin layer that can be replaced when the needles are used once. The seat of microneedles has the switching or push/pull mechanism that is associated with each set of microneedles. A user can continue to use that push/pull mechanism.

The seat of microneedles is used to detect, test and measure different biological signals. In one embodiment, the detection unit uses micro-needles that are dipped in antibody solution that changes based on the quantity of the corresponding chemical or substance in the blood. For example to detect the blood glucose, one may use the silk fibroin layer to embed the glucose oxidase.

It is known that glucose oxidase catalyzes glucose in body fluid to generate a current with intensity proportional to the glucose concentration, which translates to the glucose level in the body fluid. When the prick nanoelectronics device pushes the microneedles, piercing of the skin and blood fluid interacts with the glucose oxidase on the microneedles and reacts with glucose in the blood to generate electrons and form current. The glucose in the blood is measured accurately based on the current generated. The measurement is accurate based on equivalent blood testing results.

The invention as disclosed herein has the following advantages: (1) minimal non-invasive method to get regular results of tests indicating of actual body biological signals; (2) flexibility to multiplex microneedles with different biomarkers or antibodies to track more than one biological signal; (3) hassle free regular testing that requires minimal manual interaction; and (4) artificial intelligence based monitoring and feedback on health.

The foregoing description is only an overview of the technical solutions of the present invention, and in order to make the technical solutions of the present invention more clearly understood and to implement them in accordance with the contents of the description, the following description is made with reference to the preferred embodiments of the present invention and the accompanying detailed drawings.

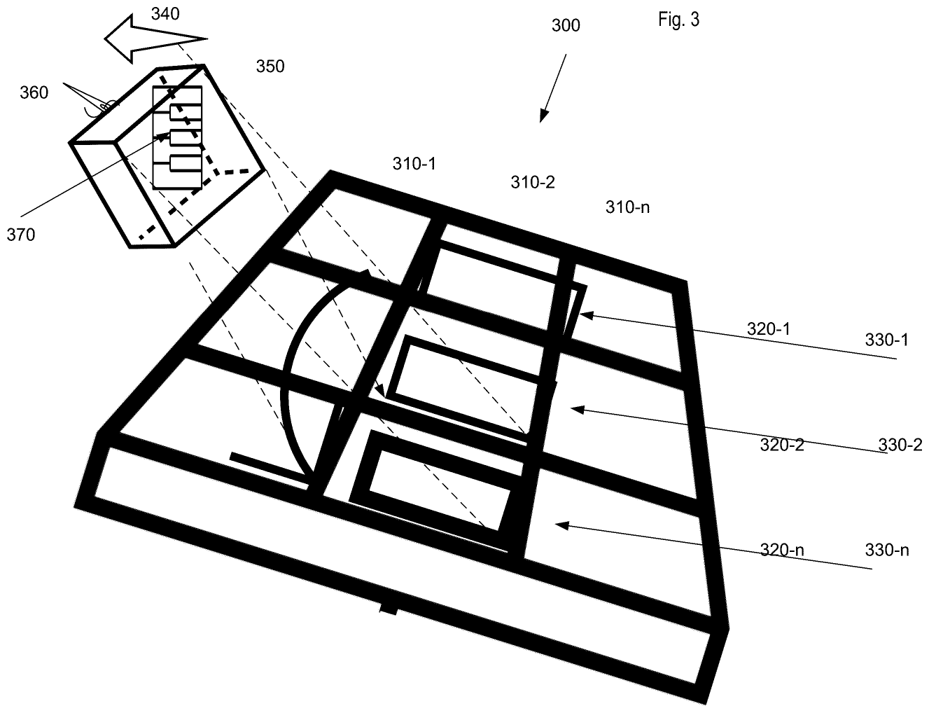

FIG. 3 is a view showing one set of microneedles 300 pressed for testing, according to one embodiment. The seat of microneedles is divided into columns 310-1, 310-2 . . . to 310-n and rows 320-1, 320-2, . . . 320-n. One set of microneedles unit is expanded as 350. The push/pull mechanism 340 is shown and exists independently for each unit. The microneedles 360 coming out of the rectangular unit can be repeated or customized based on desired frequency of testing. The internal microneedles attached to nanoelectronics on/off switch mechanism 370 is shown. For a given set of microneedles, all microneedles are pushed or pulled for testing. Not all sets have to be pressed at the same time, as shown in FIG. 3 where row 320-n and column 310-n is pressed and others are not pressed. The nanoelectronics switches have mechanism to reset and go in default off position that is not piercing the skin. The popping back in of the used set of microneedles is done seamlessly for relaxed use by the user.

In one embodiment, the prick nanoelectronics device can track the set of microneedles that have been pushed once. The prick nanoelectronics device can give indication of status of different sets of microneedles that have been used. This can be transmitted or communicated with biological signals data. The status of how many sets of microneedles is yet to be used can give a user active data as to when the seat of microneedles needs to be replaced.

A person of ordinary skill in the art would understand that different programmable push/pull nanoelectronics mechanisms exist and can be implemented here to give a customizable interface for the user. The concepts of molecular technology or molecular electronics could also be applied herein as appropriate. For example, programmable elastic buffer mechanism, nanoelectronics on-off switch or molecular technology switches can be used to get the push/pull effect for the unit of rectangular set of microneedles.

For example, 3D-printing technology could also be used to print the microneedles coated with biomarkers that operate with the seat of microneedles with push/pull mechanism for each unit independent of other units.

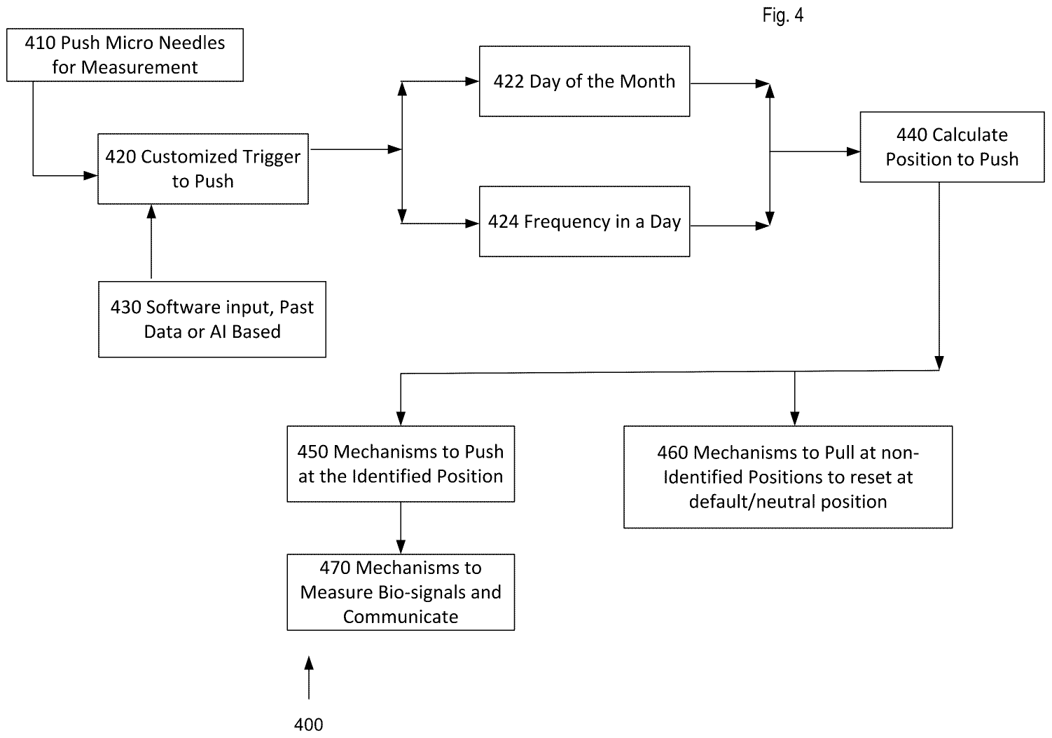

FIG. 4 is a view of customizing the prick nanoelectronics device and using the invention, according to one embodiment. 400 is a guide to designing a prick nanoelectronics device, systems and methods as disclosed herein. 410 component begins with selecting the number of microneedles required for a single test. A person of ordinary skill in the art would understand that the template of set of microneedles could be different based on the time of the day. For example, 5 microneedles could be used early in the morning, and 3 microneedles could be used in the afternoon testing. This would be appropriate if 5 different biological signals needed to be tested in the morning and only 3 different biological signals need to be tested in the afternoon. In one embodiment, the single set of microneedles is repeated and same for all days and all frequencies.

The period or interval of testing is customized by component 420. For example, some users may have serious health conditions requiring testing 5 times a day and some may be monitored in good health for athletic purposes and could go with testing 3 times a day. The frequency of testing is decided by the user. The user can input his preference using different selections as shown in 430. The user could also be given a recommendation by the system based on his past collected data 430. In one embodiment, the user could get recommendation for an artificial intelligence system 430 that performs analytics on human biological signals and has benchmarks or industry healthcare standards incorporated in its data.

The systems and methods in general would have two parameters. In one embodiment, the testing tracks one-month. One parameter day of the month 422 and another parameter 242 frequency in a day together help calculate 440 position to push on the seat of microneedles.

In one embodiment, the calculation of position could be based on an angle and a radius that is calculated based on the day of the month and frequency of testing in a day. While the disclosure here is shown using a rectangular system and rectangular units, the invention could be applied to any geometrical shape with repeatable same sized units.

Once the position to press is calculated, the corresponding nanoelectronics component is turned on to press the microneedles into the skin. A person of ordinary skill in the art would understand that different mechanical, nanoelectronics and molecular technologies could be used to design programmable on/off switches in different ways that are robust and efficient.

The mechanism to push is not permanent and can be pulled back 460 to bring the needles back into neutral or default position. Elastic buffers or springs that operate with nanoelectronics could be used here.

Once the set of microneedles are pushed, the prick nanoelectronics measures the biological signals are 470 and transmits to user's computing device. There are seamless ways to wirelessly transmit the sensor collected data to cloud applications.

A person of ordinary skill in the art would understand that a prick nanoelectronics device can be built in multiple ways with different options to put together components and functionality described herein: architectures, modules or components or mix-match some of these features. For example, a user may select his or her own architecture that includes a display and all the features of another wearable device including a smart watch. In another example, a user may select to have a simple arm band without display that interacts with his smartphone.

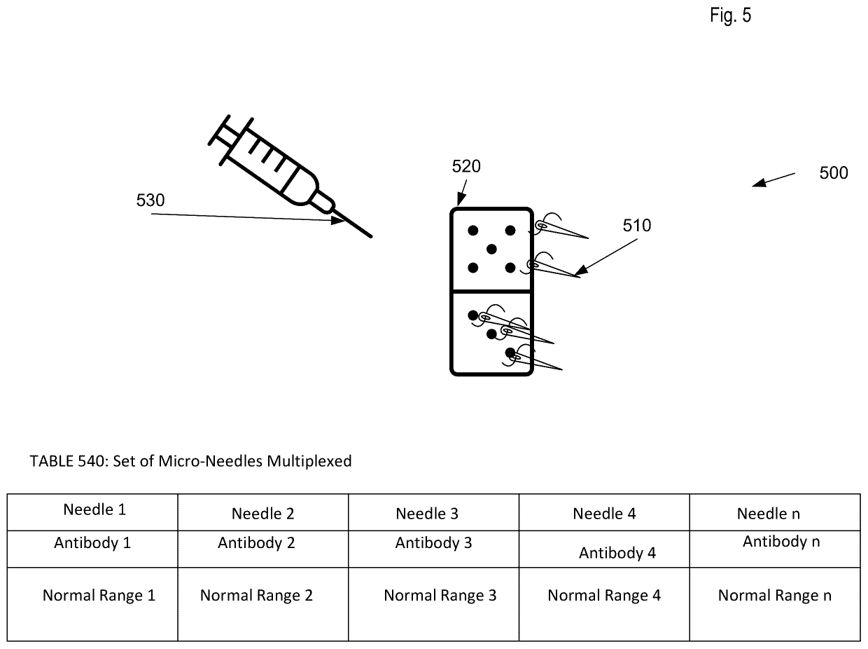

FIG. 5 is a 500 view of customizing a set of microneedles that can be repeated or varied on the seat of microneedles for the prick nanoelectronics device, according to one embodiment. 510 shows different needles that can be customized according to Table 540. The length and size of needle 1 could be completely different from that of needles 2, 3, . . . or n. Needle 1 is selected appropriately based on the biological signal to be tested and the antibody 1 or biomarker to be tested. The application or preparation of the needle with the selected biomarker or antibody is shown by 530.

The set of selected needles in a set or delineated by 520. Each needle would also have an associated normal range that triggers alarms, alerts or notifications when the biological signal measured is outside of that normal range.

A person of ordinary skill in art would appreciate that the flexibility to customize each of these depending on user's needs is an elegant solution to a user's biosensor monitoring. Notably, this is achieved in a hassle-free non-invasive manner.

The factory production of these needle sets based on a user request could be converted to a template that can be refreshed each month in a subscription/service type of model.

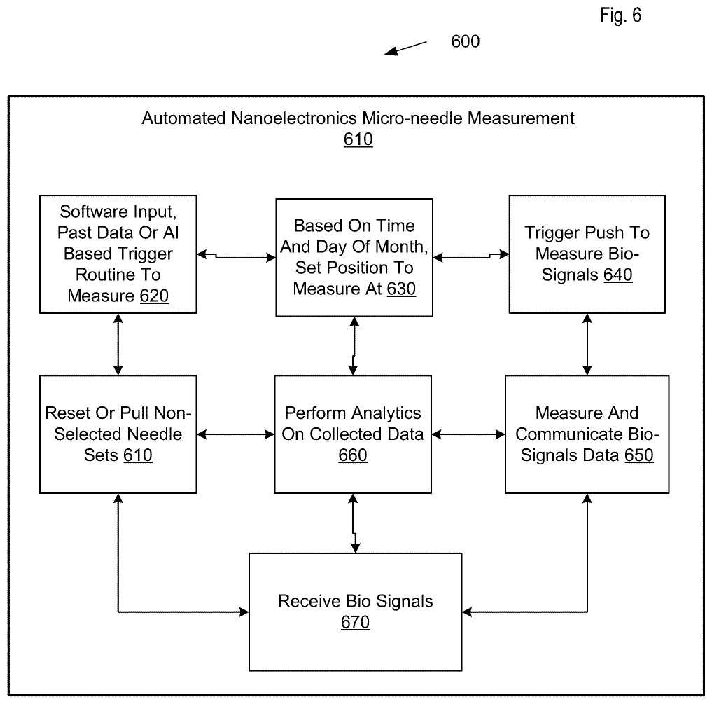

FIG. 6 is a 600 view showing design using components for the prick nanoelectronics device, according to one embodiment. 610 shows reset or pull capability for the seat of microneedles to be in rest or non-testing position. The device does not pierce the skin and sits comfortably on the desired location on a human body. 620 shows component to give software input on the desired frequency of testing, feedback based on past collected data and artificial intelligence based analytics that considers healthcare benchmarks and standards. In one embodiment, artificial intelligence-based analytics on collected biologic signals that recommends corrective steps to promote good health.

Component 630 configures the device based on the time and day of the month and desired frequency per day. A person of ordinary skill in art would understand that the component 630 is the clock/time manager for the device and will sync its time to the internet, user's computing device or a cloud service. In one embodiment, the device may have its own clock that is synced with satellite time. In another embodiment, the user could set time on the device based on his/her preference. In one embodiment, the popping out frequency is synchronized with the time stamp on the user's device including a smartphone, smartwatch, smart tablet, laptop, computer or a cloud service.

When the prick nanoelectronics device detects the time is right for a push, component 640 calculates the position for the unit of set of microneedles to be pushed and triggers the push. The push/pull mechanism is based on nanoelectronics, molecular technology components.

Component 650 measures and transmits the biological signals that are measured once the microneedles are pushed, and the skin of the user is pierced. The depth of piercing and the duration of piercing is determined based on the biological signals, biomarkers used and the needle's length, size and sensitivity.

Component 660 uses the measured biological signals data to perform analytics. In one embodiment, artificial intelligence is used to analyze predictive trends for health and concerns. Component 670 receives the data in the biological signals that can be displayed in graphical or tabular form as per user's choice. The goal of the invention is to give control to the user as to his or her biological data measured.

In a broad embodiment, the invention is systems and methods of prick nanoelectronics device allows hassle-free non-invasive regular testing and monitoring of multiplexed biological signals integrated with software/cloud-based applications customized for a user.

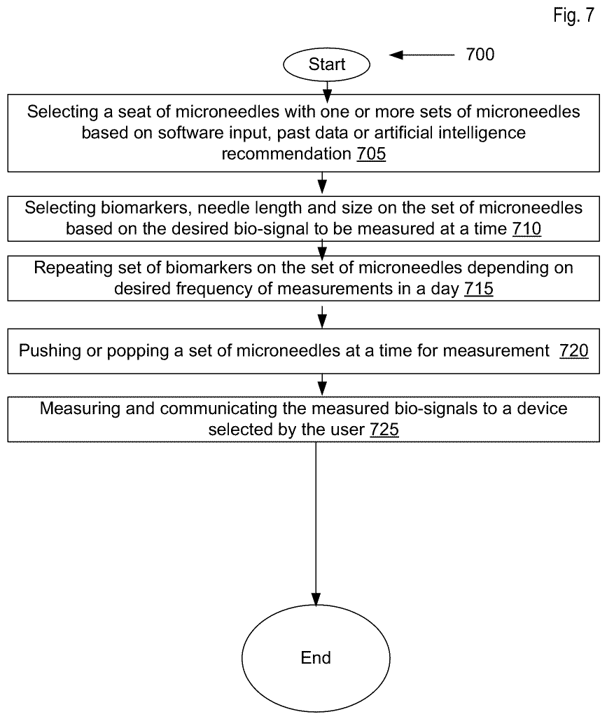

FIG. 7 is a flow diagram 700 of method of using the prick nanoelectronics device, according to one embodiment. This flowchart illustrates an embodiment wherein biological signals are monitored using multiplexed microneedles that transmit measured data including providing alarms or alerts to a user of the system when not within a predetermined range.

Referring now to FIG. 7, at block 705 a user selects a seat of microneedles that has one or

more sets of microneedles. The number of sets required can be based on software input including information on the biological signals that need to be monitored. A user's past data on biological signal can be used in analytics to give the user feedback on which biological signals are more crucial and need more monitoring than others. An artificial intelligence recommendation system could be used for the user's profile that incorporates feedback from healthcare providers and industry benchmarks or standards.

At block 710, the biomarkers are finalized with needle size and length required to achieve accurate results. This processing may include calibration processing, derivations of biological signals based on different substance or chemical concentrations, comparing received values or processed values to thresholds, computing trends, etc.

At block 715, the selected set of microneedles are repeated based on the desired frequency of testing. At block 720, the set of microneedles are automatically pushed for testing biological signals in the body based on configured parameters of testing. At block 725, the biological signals are measured and the measured data is transmitted seamlessly using wireless means to a computing device. Optionally, an alarm is triggered based at least in part on information associated with the data stream. It will be appreciated that although FIG. 7 is directed to multiple biological signals monitoring, the method described can be applied to a single biological signal including glucose monitoring.

As described above, the device can incorporate or otherwise be in communication with a single point monitor. In some embodiments, the device can be configured to display results with an attached display or remotely through display of a wireless connected device. In addition, the device can be configured to automatically transmit regular biological signals data to networked storage for access by a user, a physician or other caregiver.

The systems and methods described above have a variety of advantageous features. The data can be made available essentially immediately over the Internet or other wide-area-network to physicians based on a retrospective analysis that may occur at the recording devices, at the web server or connected device and/or on an application at the local computer (e.g., web application at a physician's office). Therefore, both prospective and retrospective analysis can be occurring at the same time, with alarm capability immediately available to the host based on the prospective processing, which data for retrospective processing and review is available to the physician or other caregiver at essentially any time.

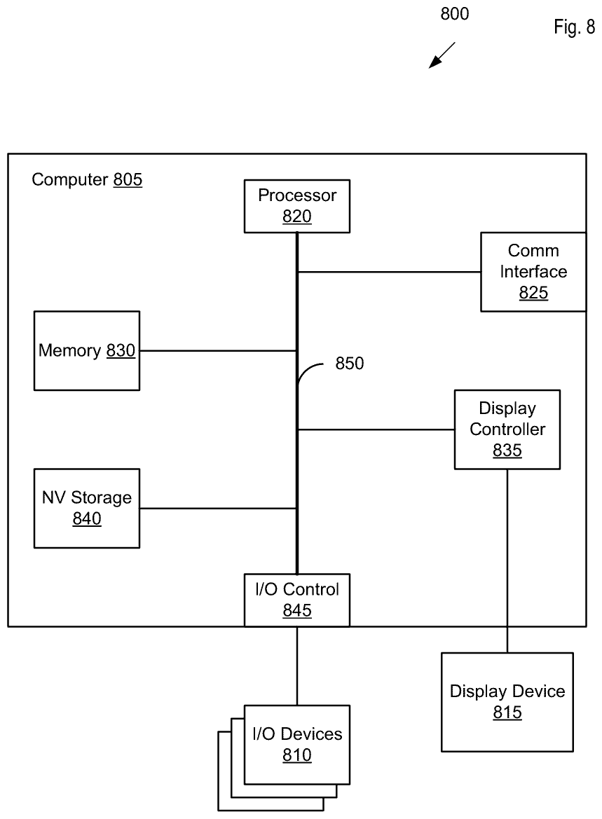

FIG. 8 shows an example of a computing device 800 on which techniques described here can be implemented and which could operate in a highly virtualized, software-defined cloud environment. The computing device 800 can be a conventional computer system or a virtual computer system that can be used as a client computer system, such as a wireless client or a workstation, or a server computer system. The computing device 800 includes a computer 805, I/O devices 810, and a display device 815. The computer 805 includes a processor 820, a communications interface 825, memory 830, display controller 835, non-volatile storage 840, and I/O controller 845. The computer 805 may be coupled to or include the I/O devices 810 and display device 815. The computing device is referenced in FIG. 1 description.

The computer 805 interfaces to external systems through the communications interface 825, which may include a modem or network interface. It will be appreciated that the communications interface 825 can be considered to be part of the computing device 800 or a part of the computer 805. The communications interface 825 can be an analog modem, integrated services for digital networks (“ISDN”) modem, cable modem, token ring interface, satellite transmission interface (e.g. “direct personal computer” also known as “direct PC”), or other interfaces for coupling a computer system to other computer systems, including virtualized interfaces and networks.

The processor 820 may be, for example, a conventional microprocessor such as an Intel Pentium microprocessor or Motorola power PC microprocessor or a virtualized processor. The memory 830 is coupled to the processor 820 by a bus 850. The memory 830 can be Dynamic Random Access Memory (DRAM) and can also include Static RAM (SRAM). The bus 850 couples the processor 820 to the memory 830, also to the non-volatile storage 840, to the display controller 835, and to the I/O controller 845, all of which may be virtual, or software-defined components.

The I/O devices 810 can include a keyboard, disk drives, printers, a scanner, and other input and output devices, including a mouse, other pointing device, voice input and recognition devices. The display controller 835 may control in the conventional manner a display on the display device 815, which can be, for example, a cathode ray tube (CRT), liquid crystal display (LCD) or other augmented reality or virtual device and/or display techniques. The display controller 835 and the I/O controller 845 can be implemented with conventional well-known technology.

The non-volatile storage 840 is often a magnetic hard disk, an optical disk, or another (possibly software-defined virtual) medium or form of storage for large amounts of data. Some of this data is often written, by a direct memory access process, into memory 830 during execution of software in the computer 805. One of skill in the art will immediately recognize that the terms “machine-readable medium” or “computer-readable medium” includes any type of storage device that is accessible by the processor 820 and also encompasses a carrier wave that encodes a data signal.

The computing device 800 is one example of many possible computer systems that have different architectures including virtualized, software-defined cloud systems and services. For example, personal computers based on an Intel microprocessor often have multiple buses, one of which can be an I/O bus for the peripherals and one that directly connects the processor 820 and the memory 830 (often referred to as a memory bus). The buses are connected together through bridge components that perform any necessary translation due to differing bus protocols.

Network and virtual computers are another type of computer system that can be used in conjunction with the teachings described here. Network computers do not usually include a hard disk or other mass storage, and the executable programs are loaded from a network connection into the memory 830 for execution by the processor 820. A Web TV system, which is known in the art, is also considered to be a computer system, but it may lack some of the components shown in FIG. 8, such as certain input or output devices. A typical computer system will usually include at least a processor, memory, and a bus coupling the memory to the processor.

Though FIG. 8 shows an example of the computing device 800, it is noted that the term “computer system,” as used here, is intended to be construed broadly as a physical computer system or a virtualized computer system and components. In general, a computer system will include a processor, memory, non-volatile storage, and an interface. A typical computer system will usually include at least a processor, memory, and a device (e.g., a bus) coupling the memory to the processor. The processor can be, for example, a general-purpose central processing unit (CPU), such as a microprocessor, or a special-purpose processor, such as a microcontroller. An example of a computer system is shown in FIG. 8.

The memory can include, by way of example but not limitation, random access memory (RAM), such as dynamic RAM (DRAM) and static RAM (SRAM). The memory can be local, remote, distributed. As used here, the term “computer-readable storage medium” is intended to include only physical media, such as memory. As used here, a computer-readable medium is intended to include all mediums that are statutory (e.g., in the United States, under 35 U.S.C. 101), and to specifically exclude all mediums that are non-statutory in nature to the extent that the exclusion is necessary for a claim that includes the computer-readable medium to be valid. Known statutory computer-readable mediums include hardware (e.g., registers, random access memory (RAM), non-volatile (NV) storage, to name a few), but may or may not be limited to hardware. Memory may also be virtualized with associated virtual software-defined components and system services.

The bus can also couple the processor to the non-volatile storage. The non-volatile storage is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. Some of this data is often written, by a direct memory access process, into memory during execution of software on the computer system. The non-volatile storage can be local, remote, or distributed. The non-volatile storage is optional because systems can be created with all applicable data available in memory.

Software is typically stored in the non-volatile storage. Indeed, for large programs, it may not even be possible to store the entire program in the memory. Nevertheless, it should be understood that for software to run, if necessary, it is moved to a computer-readable location appropriate for processing, and for illustrative purposes, that location is referred to as the memory here. Even when software is moved to the memory for execution, the processor will typically make use of hardware registers to store values associated with the software, and local cache that, ideally, serves to speed up execution. As used here, a software program is assumed to be stored at an applicable known or convenient location (from non-volatile storage to hardware registers) when the software program is referred to as “implemented in a computer-readable storage medium.” A processor is considered to be “configured to execute a program” when at least one value associated with the program is stored in a register readable by the processor.

In one example of operation, a computer system can be controlled by operating system software, which is a software program that includes a file management system, such as a disk operating system. One example of operating system software with associated file management system software is the family of operating systems known as Windows® from Microsoft Corporation of Redmond, Washington, and their associated file management systems. Another example of operating system software with its associated file management system software is the Linux operating system and its associated file management system. The file management system is typically stored in the non-volatile storage and causes the processor to execute the various acts required by the operating system to input and output data and to store data in the memory, including storing files on the non-volatile storage. The computer system may run with or without a hypervisor or containerized programs.

The bus can also couple the processor to the interface. The interface can include one or more input and/or output (I/O) devices. The I/O devices can include, by way of example but not limitation, a keyboard, a mouse or other pointing device, disk drives, printers, a scanner, and other I/O devices, including a display device. The display device can include, by way of example but not limitation, a cathode ray tube (CRT), liquid crystal display (LCD), or some other applicable known or convenient display device. The interface can include one or more of a modem or network interface. It will be appreciated that a modem or network interface can be considered to be part of the computer system. The interface can include an analog modem, isdn modem, cable modem, token ring interface, satellite transmission interface (e.g. “direct PC”), or other interfaces for coupling a computer system to other computer systems. Interfaces enable computer systems and other devices to be coupled together in a network, either physical or virtual.

FIGS. 9-16 show slides describing functionality of the Cloud Security Engine (CSE).

Several components described here, including Virtual Private/Public Clouds (VPCs), Virtual Machines (VMs), Virtual Load Balancers (VLBs), other virtualized resources and all other components and services available for deployment in a cloud, clients, servers, and engines, can be compatible with or implemented using a cloud-based computing system.

A person of ordinary skill in the art would understand that different modules or components described herein could be implemented using a cloud-based computing system. Such systems can involve a subscription for services or use a utility pricing model. Users can access the protocols of the private network through a web browser or other container application located on their client system.

A detailed description of one or more implementations of the invention is provided here along with accompanying figures that illustrate the principles of the invention. The invention is described in connection with such implementations, but the invention is not limited to any implementation. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Some portions of the detailed description are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result.

Although the foregoing implementations have been described in some detail for purposes of clarity of understanding, implementations are not necessarily limited to the details provided.

A number of embodiments have been described. Nevertheless, it will be understood that various modifications may be made without departing from the spirit and scope of the claimed invention. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

It may be appreciated that the various systems, methods, and apparatus disclosed herein may be embodied in a machine-readable medium and/or a machine accessible medium compatible with a data processing system (e.g., a computer system), and/or may be performed in any order. The structures and modules in the figures may be shown as distinct and communicating with only a few specific structures and not others. The structures may be merged with each other, may perform overlapping functions, and may communicate with other structures not shown to be connected in the figures.

The above-described functions and components may comprise instructions that are stored on a storage medium such as a computer readable medium. The instructions may be retrieved and executed by a processor. Some examples of instructions are software, program code, and firmware. Some examples of storage medium are memory devices, tapes, disks, integrated circuits, and servers. The instructions are operational when executed by the processor to direct the processor to operate in accord with some embodiments. Those skilled in the art are familiar with instructions, processor(s), and storage medium.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention. A detailed description of one or more implementations of the invention is provided here along with accompanying figures that illustrate the principles of the invention. The invention is described in connection with such implementations, but the invention is not limited to any implementation. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

The structures and modules in the figures may be shown as distinct and communicating with only a few specific structures and not others. The structures may be merged with each other, may perform overlapping functions, and may communicate with other structures not shown to be connected in the figures.

While various embodiments of the invention have been described above, they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various exemplary embodiments and implementations, the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. Thus the breadth and scope of the present disclosure should not be limited by any of the above-described exemplary embodiments.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; adjectives such as “known”, “normal”, “standard”, and terms of similar meaning should not be construed as limiting the item described to a given time period or to an item available as of a given time, but instead should be read to encompass known, normal, or standard technologies that may be available or known now or at any time in the future; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1. A method comprising an automated prick nanoelectronics device with components:
selecting a seat of microneedles comprising of microneedles including:
(i) one or more set of microneedles marked with one or more biomarkers of interest to the user; and
(ii) number of set of microneedles included on the seat based on desired frequency of testing by a user;
popping out the set of microneedles one at a time for testing based on the desired frequency of testing;
measuring the biological signals based on the applied biomarkers on the microneedles in the set of microneedles;
communicating the measured biological signals to a device selected by the user.

2. The method of claim 1, further comprising: indication of status of different sets of microneedles that have been used.

3. The method of claim 1, wherein the user can revise the set of microneedles with different biomarkers using different needle length and size based on user's health or interest.

4. The method of claim 1, further comprising: displaying the measured biological signals in a format based on user's choice including tabular, graphical, 2D, or 3D formats.

5. The method of claim 1, further comprising: alerting the user when any of the biological signals fall outside of a desired range for the user.

6. The method of claim 1, wherein: the popping out of the set of microneedles uses programmable elastic buffer mechanism, nanoelectronics on-off switch or molecular technology switches.

7. The method of claim 1, wherein: the popping out frequency is synchronized with the time stamp on the user's device including a smartphone, smartwatch, smart tablet, laptop, computer or a cloud service.

8. The method of claim 1, wherein the user can replace the seat of microneedles and the number of microneedles after use.

9. The method of claim 1, further comprising: popping back in of the used set of microneedles.

10. The method of claim 1, further comprising: artificial intelligence-based analytics on collected biologic signals that recommends corrective steps to promote good health.

11. A system comprising an automated prick nanoelectronics device with components:
a selected seat of microneedles comprising of microneedles including:
(i) one or more set of microneedles marked with one or more biomarkers of interest to the user; and
(ii) number of set of microneedles included on the seat based on desired frequency of testing by a user;
a popping out mechanism for the set of microneedles one at a time for testing based on the desired frequency of testing;
a measuring device to measure the biological signals based on the applied biomarkers on the microneedles in the set of microneedles;
a communication device to communicate the measured biological signals to a device selected by the user.

12. The system of claim 11, further comprising: indication of status of different sets of microneedles that have been used.

13. The system of claim 11, wherein the user can revise the set of microneedles with different biomarkers using different needle length and size based on user's health or interest.

14. The system of claim 11, further comprising: a display to show the measured biological signals in a format based on user's choice including tabular, graphical, 2D, or 3D formats.

15. The system of claim 11, further comprising: an alert system to alert the user when any of the biological signals fall outside of a desired range for the user.

16. The system of claim 11, the popping out of the set of microneedles uses programmable elastic buffer mechanism, nanoelectronics on-off switch or molecular technology switches.

17. The system of claim 11, wherein: the popping out frequency is synchronized with the time stamp on the user's device including a smartphone, smartwatch, smart tablet, laptop, computer or a cloud service.

18. The system of claim 11, wherein the seat of microneedles and the number of microneedles is replaced after use.

19. The system of claim 11, further comprising: popping back in of the used set of microneedles.

20. The system of claim 11, further comprising: artificial intelligence-based analytics on collected biologic signals that recommends corrective steps to promote good health.
